(19) United States
(12) Patent Application Publication
(10) Pub. No.: US 2025/0352643 A1
(43) Pub. Date: Nov. 20, 2025

(54) Device for Collecting Airborne Allergens in Water

(71) Applicants: Christian Solomon, Georgetown, TX (US); Emily Dawn Solomon, Georgetown, TX (US)

(72) Inventors: Christian Solomon, Georgetown, TX (US); Emily Dawn Solomon, Georgetown, TX (US)

(21) Appl. No.: 18/665,628

(22) Filed: May 16, 2024

(51) Int. Cl. A61K 39/36; A61K 39/00; B01D 47/02; C02F 1/68

(52) U.S. Cl. CPC A61K 39/36; B01D 47/02; C02F 1/68; A61K 2039/542

(57) ABSTRACT

The Device for Collecting Airborne Allergens in Water is an invention which uses drinking water to capture airborne allergens present in the consumer's immediate environment. The device accomplishes this by directing air flow through a screen onto the surface of water in a drinking cup, thereby trapping the allergens on the surface of the water. The device can be placed in a location with natural wind currents or be used in conjunction with a fan to direct air flow into the device. The device provides the consumer with a cup of water containing locally present allergens, which is to be consumed daily. When the water (containing local allergens) is regularly consumed, the consumer will gradually build immunity to allergens present in his/her specific environment.

### BACKGROUND/SUMMARY

In 1817, Dr. John Bostock gave up his medical practice and relocated to London, England, for scientific research. In 1819, Bostock was perhaps the first to describe “hay fever,” which likely afflicted him personally. During the subsequent nine years, Bostock described another 28 cases of hay fever. In the late 1820's, as hay fever cases began to emerge in London, a public outcry over contaminated drinking water broke out in the city. London's public water source, the Thames River, had become little more than an open sewer. London's public water crises likely spurred the work of inventor James Simpson. Simpson, the engineer for the Chelsea Water Works, developed an experimental filter which filtered 90,000 gallons of water a day. By 1830, Simpson's experimental filter had proven successful, and the Chelsea Water Works Company installed a permanent water filter. This filter provided a clean filtered water source to a large section of London. Since then, public water filtration has become the norm in municipalities and has likely saved the lives of millions from disease.

However, there seems to be a negative effect to filtering drinking water. It is my belief that the emergence of hay fever cases in London in the early 1800s began due to the aversion of drinking polluted water from the Thames River. The city's solution to the polluted water was a public water filtration system that removed the source of many diseases, but it also removed pollens present in the local environment. As lives were saved with filtered drinking water, cities all over the world began to filter public drinking water. Now that most people only drink filtered water, they have lost one of the primary benefits of drinking surface water, which is immunity to seasonal allergies in their immediate environment. This occurs because when one drinks unfiltered surface water (the source of water for most of human history), one drinks pollens that are present in the air around the water source and become trapped on the surface of the water. Regularly drinking pollens present in one's immediate environment is a natural means of immunotherapy for seasonal allergies. Alternatively, people who only drink filtered water do not develop natural immunity to airborne allergens. It is my belief that by regularly drinking clean filtered water, in which pollens present in the immediate environment are reintroduced, in time one will develop immunity to seasonal allergies. Therefore, we can have our cake and eat it too. By using daily use of the Device for Collecting Airborne Allergens in Water we can drink water free of disease and pollutants, but also enjoy the benefit of immunity to allergens present in our local environment.

### BRIEF DESCRIPTION OF DRAWINGS

FIG. 1 is a front view of the exterior of the invention.

FIG. 2 is a side view of the exterior of the invention.

FIG. 3 is a top-down perspective view of the inside of the invention, which contains four cups each containing potable water.

### DETAILED DESCRIPTION OF THE INVENTION

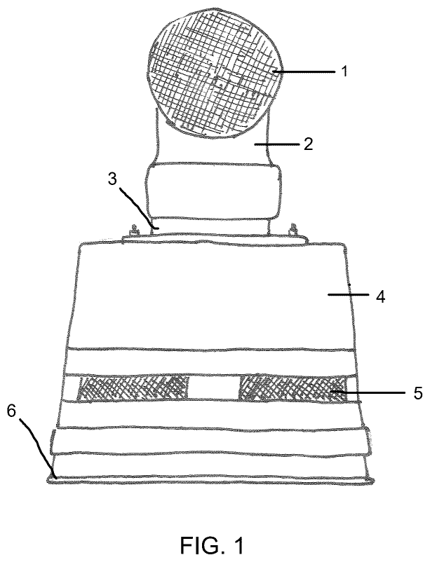

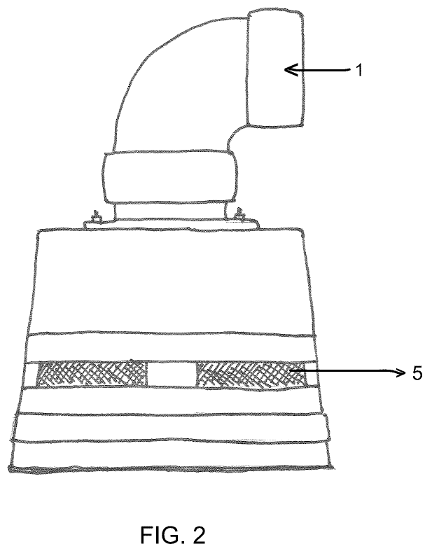

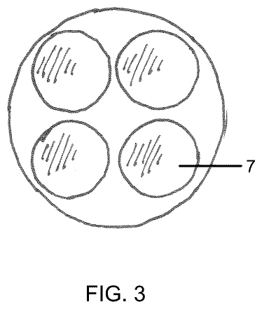

The invention is a Device for Collecting Airborne Allergens in Water, which enables a user to capture airborne allergens present in the immediate environment of the device in drinking water. As can be seen in FIG. 1 through 3, the invention comprises an airway inlet 2 attached by way of a flange 3 to a container body 4 with multiple airway outlet screens 5 and an enclosed base 6. As can be seen in FIG. 3, one or multiple cups (containing potable water) 7 are placed on the base 6, within the container body 4. The invention is designed to be placed in a location where airflow naturally blows into the airway inlet 2 or can be used in conjunction with a variable speed fan to direct airflow into the airway inlet 2. As air flows into the invention, it passes through the airway inlet screen 1, which keeps insects and larger debris out of the invention. The airway inlet 2 is bent at a ninety-degree angle downward, which forces airflow onto the surface of the water contained in the cup/cups 7, sitting on the base. As the airflow makes contact with the water in the cup/cups 7, airborne allergens are transferred from the air onto the surface of the water. The airflow then departs the invention through the airway outlet screens 5 around the perimeter of the container body 4. The airway outlet screens 5 are situated lower on the container body 4 than the top of the cup/cups 7, within the container body 4. Therefore, the majority of the airflow will not bypass the water within the cup/cups 7. The invention is designed to be in constant use, with the user removing the cup/cups 7 of water daily for consumption and replacing them with a new cup/cups 7 of potable water. The invention as diagramed in FIG. 1 through 3 is scaled for use by a single individual or household, but can be upscaled for industrial or municipal usage.

What is claimed is:

1. A device for collecting airborne allergens in water comprising:
an airway inlet (with one end covered by a screen) connected by a flange to an opening in a container body;
the container body has multiple airway outlets (covered by screens), sits on an enclosed base, and contains one or more cups of fluid (which are open on top).
